(19) United States
(12) Patent Application Publication
(10) Pub. No.: US 2010/0184065 A1
(43) Pub. Date: Jul. 22, 2010

(54) Method of Predicting Non-Response to First Line Chemotherapy

(75) Inventors: Timothy J. Yeatman, Thonotosassa, FL (US); Deepak Agrawal, Tampa, FL (US); Adil Daud, Tampa, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 12/685,367

(22) Filed: Jan. 11, 2010

Related U.S. Application Data
(63) Continuation of application No. PCT/US2008/069649, filed on Jul. 10, 2008.
(60) Provisional application No. 60/948,817, filed on Jul. 10, 2007.

(51) Int. Cl. C12Q 1/68

(52) U.S. Cl. 435/6

(57) ABSTRACT

The invention provides a method for determining a prognosis of colorectal cancer in a colorectal cancer patient, comprising classifying said patient as having a good prognosis or a poor prognosis using measurements of a plurality of gene products in a cell sample taken from said patient, said gene products being respectively products of at least 1 of the genes listed in Table 1, or respective functional equivalents thereof, wherein said good prognosis predicts a positive response to standard chemotherapy regimens, and said poor prognosis predicts non-responsiveness. Provided herein, the invention includes a gene signature to predict which patients will to benefit from standard colon cancer therapy; alternatively, patients who are classified as non-responders may be more likely to benefit from a novel agent such as a Notch inhibitor.

### CROSS REFERENCE TO RELATED APPLICATIONS

This application is a continuation of prior filed International Application Serial No. PCT/US2008/069649 filed Jul. 10, 2008, which claims priority to U.S. Provisional Application No. 60/948,817, filed Jul. 10, 2007.

### GOVERNMENT SUPPORT

This invention was made with Government support under Grant No. 5R21CA101355-02 awarded by the National Institutes of Health. The Government has certain rights in the invention.

### FIELD OF THE INVENTION

The invention relates to molecular markers that can be used for prognosis of colorectal cancer. The invention also relates to methods and computer systems for determining a prognosis of colorectal cancer in a colorectal cancer patient based on the molecular markers. The invention also relates to methods and computer systems for determining chemotherapy for a colorectal cancer patient and for enrolling patients in clinical trials.

### BACKGROUND OF THE INVENTION

Ranked as the third most commonly diagnosed cancer and the second leading cause of cancer deaths in the United States (American Cancer Society, “Cancer facts and figures,” Washington, D.C.: American Cancer Society (2000)), colon cancer is a deadly disease afflicting nearly 130,000 new patients yearly in the United States. Colon cancer is the only cancer that occurs with approximately equal frequency in men and women. There are several potential risk factors for the development of colon and/or rectal cancer. Known factors for the disease include older age, excessive alcohol consumption, sedentary lifestyle (Reddy, Cancer Res., 41:3700-3705 (1981)), and genetic predisposition (Potter J Natl Cancer Institute, 91:916-932 (1999)).

Several molecular pathways have been linked to the development of colon cancer (see, for example, Leeman et al., J. Pathol., 201(4):528-34 (2003); Kanazawa et al., Tumori., 89(4):408-11 (2003); and Notarnicola et al., Oncol Rep., 10(6): 1987-91 (2003)), and the expression of key genes in any of these pathways may be affected by inherited or acquired mutation or by hypermethylation. A great deal of research has been performed with regard to identifying genes for which changes in expression may provide an early indicator of colon cancer or a predisposition for the development of colon cancer. Unfortunately, no research has yet been conducted on identifying specific genes associated with colorectal cancer and specific outcomes to provide an accurate prediction of prognosis.

Survival of patients with colon and/or rectal cancer depends to a large extent on the stage of the disease at diagnosis. Devised nearly seventy years ago (Dukes, 1932, J Pathol Bacteriol 35:323), the modified Dukes' staging system for colon cancer, discriminates four stages (A, B, C, and D), primarily based on clinicopathologic features such as the presence or absence of lymph node or distant metastases. Specifically, colonic tumors are classified by four Dukes' stages: A, tumor within the intestinal mucosa; B, tumor into muscularis mucosa; C, metastasis to lymph nodes and D, metastasis to other tissues. Of the systems available, the Dukes' staging system, based on the pathological spread of disease through the bowel wall, to lymph nodes, and to distant organ sites such as the liver, has remained the most popular. Despite providing only a relative estimate for cure for any individual patient, the Dukes' staging system remains the standard for predicting colon cancer prognosis, and is the primary means for directing adjuvant therapy.

The Dukes' staging system, however, has only been found useful in predicting the behavior of a population of patients, rather than an individual. For this reason, any patient with a Dukes A, B, or C lesion would be predicted to be alive at 36 months while a patient staged as Dukes D would be predicted to be dead. Unfortunately, application of this staging system results in the potential over-treatment or under-treatment of a significant number of patients. Further, Dukes' staging can only be applied after complete surgical resection rather than after a pre-surgical biopsy.

DNA array technologies have made it possible to monitor the expression level of a large number of genetic transcripts at any one time (see, e.g., Schena et al., 1995, Science 270:467-470; Lockhart et al., 1996, Nature Biotechnology 14:1675-1680; Blanchard et al., 1996, Nature Biotechnology 14:1649; Ashby et al., U.S. Pat. No. 5,569,588, issued Oct. 29, 1996). Of the two main formats of DNA arrays, spotted cDNA arrays are prepared by depositing PCR products of cDNA fragments with sizes ranging from about 0.6 to 2.4 kb, from full length cDNAs, ESTs, etc., onto a suitable surface (see, e.g., DeRisi et al., 1996, Nature Genetics 14:457-460; Shalon et al., 1996, Genome Res. 6:689-645; Schena et al., 1995, Proc. Natl. Acad. Sci. U.S.A. 93:10539-11286; and Duggan et al., Nature Genetics Supplement 21:10-14). Alternatively, high-density oligonucleotide arrays containing thousands of oligonucleotides complementary to defined sequences, at defined locations on a surface are synthesized in situ on the surface by, for example, photolithographic techniques (see, e.g., Fodor et al., 1991, Science 251:767-773; Pease et al., 1994, Proc. Natl. Acad. Sci. U.S.A. 91:5022-5026; Lockhart et al., 1996, Nature Biotechnology 14:1675; McGall et al., 1996, Proc. Natl. Acad. Sci U.S.A. 93:13555-13560; U.S. Pat. Nos. 5,578,832; 5,556,752; 5,510,270; and 6,040,138). Methods for generating arrays using inkjet technology for in situ oligonucleotide synthesis are also known in the art (see, e.g., Blanchard, International Patent Publication WO 98/41531, published Sep. 24, 1998; Blanchard et al., 1996, Biosensors and Bioelectronics 11:687-690; Blanchard, 1998, in Synthetic DNA Arrays in Genetic Engineering, Vol. 20, J. K. Setlow, Ed., Plenum Press, New York at pages 111-123).

By simultaneously monitoring tens of thousands of genes, microarrays have permitted identification of biomarkers of cancer (Welsh et al., PNAS, 100(6):3410-3415 (March 2003)), creating gene expression-based classifications of cancers (Alzadeh et al., Nature, 403:513-11 (2000); and Garber et al., Proc Natl Acad Sci USA, 98:13784-9 (2001); development of gene based multi-organ cancer classifiers (Bloom et al, Am J Pathol 164:9-16, 2004; Giordano et al., Am J Pathol, 159:1231-8 (2001); Ramaswamy et al., Proc Natl Acad Sci USA, 98:15149-54 (2001); and Su et al., Cancer Res, 61:7388-93 (2001)), identification of tumor subclasses (Dyrskjot et al., Nat Genet, 33:90-6 (2003); Bhattacharjee et al., Proc Natl Acad Sci USA, 98:13790-5 (2001); Garber et al., Proc Natl Acad Sci USA, 98:13784-9. (2001); and Sorlie et al., Proc Natl Acad Sci USA, 98:10869-74 (2001)), discovery of progression markers (Sanchez-Carbayo et al., Am J Pathol, 163:505-16 (2003); and Frederiksen et al., J Cancer Res Clin Oncol, 129:263-71 (2003)); and prediction of disease outcome (Henshall et al., Cancer Res, 63:4196-203 (2003); Shipp et al., Nat Med, 8:68-74 (2002); Beer et al., Nat Med, 8:816-24 (2002); Pomeroy et al., Nature, 415:436-42 (2002); van't Veer et al., Nature, 415:530-6 (2002); Vasselli et al., Proc Natl Acad Sci USA, 100:6958-63 (2003); Takahashi et al., Proc Natl Acad Sci USA, 98:9754-9 (2001); WO 2004/065545 A2; WO 02/103320 A2)); and in drug discovery (Marton et al., Nat Med, 4(11):1293-301 (1998); and Gray et al., Science, 281:533-538 (1998)).

One tool that has been applied to microarrays to decipher and compare genome expression patterns in biological systems is Significance Analysis of Microarrays, or SAM (Tusher et al., 2001, Proc. Natl. Acad. Sci. 98:5116-5121). This statistical method was developed as a cluster tool for use in identifying genes with statistically significant changes in expression. SAM has been used for a variety of purposes, including identifying potential drugs that would be effective in treating various conditions associated with specific gene expressions (Bunney et al., Am J Psychiatry, 160(4):657-66 (April 2003)).

Sophisticated and powerful machine learning algorithms have been applied to transcriptional profiling analysis. For example, a modified “Fisher classification” approach has been applied to distinguish patients with good prognosis from those who do not have a good prognosis, based on their expression profiles (van't Veer et al., 2002, Nature 415: 530-6). A similar study has been reported using an artificial neural network (Bloom et al, Am J Pathol 164:9-16, 2004; Khan et al., 2001, Nat Med 7: 673-9). Support Vector Machine (SVM) (see, e.g., Brown et al., Proc. Natl. Acad. Sci. 97(1):262-67 (2000); Zien et al., Bioinformatics, 16(9):799-807 (2000); Furey et al., Bioinformatics, 16(10):906-914 (2000)) is a correlation tool shown to perform well in multiple areas of biological analysis, including evaluating microarray expression data (Brown et al, Proc Natl Acad Sci USA, 97:262-267 (2000)), detecting remote protein homologies (Jaakkola et al., Proceedings of the 7.sup.th International Conference on Intelligent Systems for Molecular Biology, AAAI Press, Menlo Park, Calif. (1999)), and classification of cancer tissues (Furey et al., Bioinformatics, 16(10):906-914 (2000)). Furey describes using SVM to classify colon cancer tissues based on expression levels of a set of 2000 genes or a set of 1000 genes having the highest minimal intensity across 60 colon tissue samples (40 tumors and 22 normal tissues) on an Affymetrix® oligonucleotide microarray.

Wang et al. (Wang et al., 2004, J. Clinical Oncology 22:1564-1571) reported identification of a 60-gene and a 23-gene signature for prediction of cancer recurrence in Dukes' B patients using an Affymetrix® U133a GeneChip. This signature was validated in 36 independent patients. Two supervised class prediction approaches were used to identify gene markers that could best discriminate between patients who would experience relapse and patients who would remain disease-free. A multivariate Cox model was built to predict recurrence. The overall performance accuracy was reported as 78%.

Resnick et al. (Resnick et al., 2004, Clin. Can. Res. 10:3069-3075) reported a study of the prognostic value of epidermal growth factor receptor, c-MET, b-catenin, and p53 protein expression in TNM stage II colon cancer using tissue microarray technology.

Muro et al. (Muro et al., 2003, Genome Biology 4:R21) describes identification and analysis of the expression levels of 1,536 genes in colorectal cancer and normal tissues using a parametric clustering method. Three groups of genes were discovered. Some of the genes were shown to not only correlate with the differences between tumor and normal tissues but also the presence and absence of distant metastasis.

Discussion or citation of a reference herein shall not be construed as an admission that such reference is prior art to the present invention.

### SUMMARY OF INVENTION

The invention provides a method for determining a prognosis of colorectal cancer in a colorectal cancer patient, comprising classifying said patient as having a good prognosis or a poor prognosis using measurements of a plurality of gene products in a cell sample taken from said patient, said gene products being respectively products of at least 1 of the genes listed in Table 1, or respective functional equivalents thereof, wherein said good prognosis predicts a positive response to standard chemotherapy regimens, and said poor prognosis predicts non-responsiveness.

### BRIEF DESCRIPTION OF THE DRAWINGS

For a fuller understanding of the invention, reference should be made to the following detailed description, taken in connection with the accompanying drawings, in which:

FIG. 1A is a graph showing the distribution of composite values of the average expression values for 3 genes involved in apoptosis downstream of DNA damage (DD).

FIG. 1B is a graph showing the distribution of composite values of the average expression values for 3 genes involved in apoptosis downstream of DNA damage (DD).

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

The invention provides markers, i.e., genes, the expression levels of which discriminate between a good prognosis and a poor prognosis for patients with colorectal cancer. As used herein, a good prognosis predicts which patients will to benefit from standard colon cancer therapy; alternatively, patients who are classified as non-responders may be more likely to benefit from a novel agent such as a Notch inhibitor.

The identities of these markers and the measurements of their respective gene products, e.g., measurements of levels (abundances) of their encoded mRNAs or proteins, can be used by application of a pattern recognition algorithm to develop a prognosis predictor that discriminates between a good and poor prognosis in colorectal cancer using measurements of such gene products in a sample from a patient.

Colorectal cancer includes colon cancer and rectal cancer. Such molecular markers, the expression levels of which can be used for prognosis of colorectal cancer in a colorectal cancer patient, are listed in Table 1, infra. Measurements of gene products of these molecular markers, as well as of their functional equivalents, can be used for prognosis of colorectal cancer. A functional equivalent with respect to a gene, designated as gene A, refers to a gene that encodes a protein or mRNA that at least partially overlaps in physiological function in the cell to that of the protein or mRNA encoded by gene A. In particular, prognosis of colorectal cancer in a colorectal cancer patient is carried out by a method comprising classifying the patient as having a good or poor prognosis based on a profile of measurements (e.g., of the levels) of gene products of (i.e., encoded by) at least some of the genes in Table 1, or functional equivalents of such genes; or of at least 2%, 5%, 10%, 20%, 30%, 40%, 50%, 60%, 70%, 80%, or 90% of the genes in Table 1, or functional equivalents of such genes or functional equivalents of such genes, in an appropriate cell sample from the patient, e.g., a tumor cell sample obtained from biopsy or after surgical resection.

Preferably, the tumor sample is contaminated with less than 50%, 40%, 30%, 20%, or 10% of normal cells. Such a profile of measurements is also referred to herein as an “expression profile.” In some embodiments, “at least some of the genes listed” in a table refers to at least, 4 or 6 of the genes listed in the table. In other embodiments, all genes from Table 1 are used. Different subcombinations of genes from Table 1 may be used as the marker set to carry out the prognosis methods of the invention.

In a specific embodiment, the classifying of the patient as having good or poor prognosis is carried out using measurements of gene products of about 9 total genes, in which all or at least 20%, 30%, 40%, 50%, 60%, 70%, 80%, or 90% of the genes are from Table 1 or their functional equivalents.

The measurements in the profiles of the gene products that are used can be any suitable measured values representative of the expression levels of the respective genes. The measurement of the expression level of a gene can be direct or indirect, e.g., directly of abundance levels of RNAs or proteins or indirectly, by measuring abundance levels of cDNAs, amplified RNAs or DNAs, proteins, or activity levels of RNAs or proteins, or other molecules (e.g., a metabolite) that are indicative of the foregoing. In one embodiment, the profile comprises measurements of abundances of the transcripts of the marker genes. The measurement of abundance can be a measurement of the absolute abundance of a gene product. The measurement of abundance can also be a value representative of the absolute abundance, e.g., a normalized abundance value (e.g., an abundance normalized against the abundance of a reference gene product) or an averaged abundance value (e.g., average of abundances obtained at different time points or from different tumor cell samples from the patients, or average of abundances obtained using different probes, etc.), or a combination of both. As an example, the measurement of abundance of a gene transcript can be a value obtained using an Affymetrix® GeneChip® to measure hybridization to the transcript.

In another embodiment, the expression profile is a differential expression profile comprising differential measurements of a plurality of transcripts in a sample derived from the patient versus measurements of the plurality of transcripts in a reference sample, e.g., a cell sample of normal cells. Each differential measurement in the profile can be but is not limited to an arithmetic difference, a ratio, or a log (ratio). As an example, the measurement of abundance of a gene transcript can be a value for the transcript obtained using a cDNA array in a two-color measurement. The invention also provides methods and systems for predicting prognosis of colorectal cancer in a colorectal cancer patient based on a measured marker profile comprising measurements of the markers of the present invention, e.g., an expression profile comprising measurements of transcripts of at least some of the genes listed in Table 1, or functional equivalents of such genes. The methods and systems of the invention use a prognosis predictor (also termed herein a “classifier”) for predicting prognosis. The prognosis predictor can be based on any appropriate pattern recognition method that receives an input comprising a marker profile and provides an output comprising data indicating a good prognosis or a poor prognosis. The prognosis predictor is trained with training data from a plurality of colorectal cancer patients for whom marker profiles and prognosis outcomes are known. The plurality of patients used for training the prognosis predictor is also referred to herein as the training population. The training data comprise for each patient in the training population (a) a marker profile comprising measurements of gene products of a plurality of genes, respectively, in an appropriate cell sample, e.g., a tumor cell sample, taken from the patient; and (b) prognosis outcome information (i.e., information regarding whether or not survival occurred over a predetermined time period, for example, from diagnosis or from surgical resection of the cancer).

Various prognosis predictors can be used in conjunction with the present invention. In preferred embodiments, an artificial neural network or a support vector machine is used as the prognosis predictor. In some embodiments, additional patients having known marker profiles and prognosis outcomes can be used to test the accuracy of the prognosis predictor obtained using the training population. Such additional patients are also called “the testing population.”

The markers in the marker sets are selected based on their ability to discriminate prognosis of colorectal cancer in a plurality of colorectal cancer patients for whom the prognosis outcomes are known. Various methods can be used to evaluate the correlation between marker levels and cancer prognosis. For example, genes whose expression levels are significantly different in tumor samples from patients who exhibit good prognosis and in tumor samples from patients who exhibit poor prognosis can be identified using an appropriate statistical method, e.g., t-test or significance analysis of microarray (SAM).

Diagnostic and Prognostic Marker Sets

The invention provides molecular marker sets (of genes) that can be used for prognosis of colorectal cancer in a colorectal cancer patient based on a profile of the markers in the marker set (containing measurements of marker gene products). Table 1 lists markers that can be used to discriminate between good and poor prognosis of colorectal cancer according to the method of the invention.

In preferred embodiments, the methods of the invention use a prognosis predictor, also called a classifier, for predicting prognosis. The prognosis predictor can be based on any appropriate pattern recognition method that receives an input comprising a marker profile and provides an output comprising data indicating a good prognosis or a poor prognosis. The prognosis predictor is trained with training data from a training population of colorectal cancer patients. Typically, the training data comprise for each of the colorectal cancer patients in the training population a marker profile comprising measurements of respective gene products of a plurality of genes in a tumor cell sample taken from the patient and prognosis outcome information. In a preferred embodiment, the training population comprises patients from each of the different stages of colorectal cancer, e.g., from adenomas (precancerous polyps), and Dukes stages A, B, C, and D. In another preferred embodiment, the training population comprises patients from each of the different TNM stages of colorectal cancer.

In a preferred embodiment, the prognosis predictor is an artificial neural network (ANN). An ANN can be trained with the training population using any suitable method known in the art. In a specific embodiment, the ANN is a feed-forward back-propagation neural network with a single hidden layer of 10 units, a learning rate of 0.05, and a momentum of 0.2.

In still other embodiments, the prognosis predictor can also be based on other classification (pattern recognition) methods, e.g., logic regression, linear or quadratic discriminant analysis, decision trees, clustering, principal component analysis or nearest neighbor classifer analysis. Such prognosis predictors can be trained with the training population using methods described in the relevant sections, infra. The marker profile can be obtained by measuring the plurality of gene products in a tumor cell sample from the patient using a method known in the art.

In a specific embodiment, the prognosis method of the invention can be used for evaluating whether a colorectal cancer patient may benefit from chemotherapy. The benefit of adjuvant chemotherapy for colorectal cancer appears limited to patients with Dukes stage C disease where the cancer has metastasized to lymph nodes at the time of diagnosis. For this reason, the clinicopathological Dukes' staging system is critical for determining how adjuvant therapy is administered. Unfortunately, as noted above, Dukes' staging is not very accurate in predicting overall survival and thus its application likely results in the treatment of a large number of patients to benefit an unknown few. Alternatively, there are a number of patients who would benefit from therapy that do not receive it based on the Dukes' staging system. Accordingly, an important use of the prognosis/survival classifier of the present invention is the ability to identify those Dukes' stage B and C cases for which chemotherapy may be beneficial.

Thus, in one embodiment, the invention provides a method for evaluating whether a colorectal cancer patient should be treated with chemotherapy, comprising (a) classifying said patient as having a good prognosis or a poor prognosis using a method described above; and (b) determining that said patient's predicted response favors treatment of the patient with chemotherapy, or an alternative treatment wherein the patient has a poor prognosis. In one embodiment, the patient is further staged using Dukes staging.

Sample Collection

In the present invention, gene products, such as target polynucleotide molecules or proteins, are extracted from a sample taken from an individual afflicted with colorectal cancer. The sample may be collected in any clinically acceptable manner, but must be collected such that marker-derived polynucleotides (i.e., RNA) are preserved (if gene expression is to be measured) or proteins are preserved (if encoded proteins are to be measured). In one embodiment, samples can be microdissected (>80% tumor cells) by frozen section guidance and RNA extraction performed using Trizol followed by secondary purification on RNAEasy columns In another embodiment, samples can be paraffin-embedded tissue sections (see, e.g., U.S. Patent Application Publication No. 2005/0048542A1, which is incorporated by reference herein in its entirety). The mRNA profiles of paraffin-embedded tissue samples are preferably obtained using quantitative reverse transcriptase polymerase chain reaction qRT-PCR.

In a specific embodiment, mRNA or nucleic acids derived therefrom (i.e., cDNA or amplified RNA or amplied DNA) are preferably labeled distinguishably from polynucleotide molecules of a reference sample, and both are simultaneously or independently hybridized to a microarray comprising some or all of the markers or marker sets or subsets described above. Alternatively, mRNA or nucleic acids derived therefrom may be labeled with the same label as the reference polynucleotide molecules, wherein the intensity of hybridization of each at a particular probe is compared.

A sample may comprise any clinically relevant tissue sample, such as a tumor biopsy or fine needle aspirate, or a sample of body fluid, such as blood, plasma, serum, lymph, ascitic fluid, cystic fluid, or urine. The sample may be taken from a human, or, in a veterinary context, from non-human animals such as ruminants, horses, swine or sheep, or from domestic companion animals such as felines and canines.

Methods for preparing total and poly(A)+RNA are well known and are described generally in Sambrook et al., MOLECULAR CLONING—A LABORATORY MANUAL (2ND ED.), Vols. 1-3, Cold Spring Harbor Laboratory, Cold Spring Harbor, N.Y. (1989)) and Ausubel et al., CURRENT PROTOCOLS IN MOLECULAR BIOLOGY, vol. 2, Current Protocols Publishing, New York (1994)). Preferably, total RNA, or total mRNA (poly(A)+RNA) is measured in the methods of the invention directly or indirectly (e.g., via measuring cDNA or cRNA).

RNA may be isolated from eukaryotic cells by procedures that involve lysis of the cells and denaturation of the proteins contained therein. Cells of interest include wild-type cells (i.e., non-cancerous), drug-exposed wild-type cells, tumor- or tumor-derived cells, modified cells, normal or tumor cell line cells, and drug-exposed modified cells. Preferably, the cells are breast cancer tumor cells.

Additional steps may be employed to remove DNA. Cell lysis may be accomplished with a nonionic detergent, followed by microcentrifugation to remove the nuclei and hence the bulk of the cellular DNA. In one embodiment, RNA is extracted from cells of the various types of interest using guanidinium thiocyanate lysis followed by CsCl centrifugation to separate the RNA from DNA (Chirgwin et al., Biochemistry 18:5294-5299 (1979)). Poly(A)+RNA is selected by selection with oligo-dT cellulose (see Sambrook et al., MOLECULAR CLONING—A LABORATORY MANUAL (2ND ED.), Vols. 1-3, Cold Spring Harbor Laboratory, Cold Spring Harbor, N.Y. (1989). Alternatively, separation of RNA from DNA can be accomplished by organic extraction, for example, with hot phenol or phenol/chloroform/iso amyl alcohol.

If desired, RNase inhibitors may be added to the lysis buffer Likewise, for certain cell types, it may be desirable to add a protein denaturation/digestion step to the protocol.

For many applications, it is desirable to preferentially enrich mRNA with respect to other cellular RNAs, such as transfer RNA (tRNA) and ribosomal RNA (rRNA). Most mRNAs contain a poly(A) tail at their 3′ end. This allows them to be enriched by affinity chromatography, for example, using oligo(dT) or poly(U) coupled to a solid support, such as cellulose or Sephadex™ (see Ausubel et al., CURRENT PROTOCOLS IN MOLECULAR BIOLOGY, vol. 2, Current Protocols Publishing, New York (1994). Once bound, poly(A)+mRNA is eluted from the affinity column using 2 mM EDTA/0.1% SDS.

In a specific embodiment, total RNA or total mRNA from cells is used in the methods of the invention. The source of the RNA can be cells of an animal, e.g., human, mammal, primate, non-human animal, dog, cat, mouse, rat, bird, etc. In specific embodiments, the method of the invention is used with a sample containing total mRNA or total RNA from 1×10⁶ cells or less. In another embodiment, proteins can be isolated from the foregoing sources, by methods known in the art, for use in expression analysis at the protein level.

Probes to the homologs of the marker sequences disclosed herein can be employed preferably when non-human nucleic acid is being assayed.

Determination of Abundance Levels of Gene Products

The abundance levels of the gene products of the genes in a sample may be determined by any means known in the art. The levels may be determined by isolating and determining the level (i.e., amount) of nucleic acid transcribed from each marker gene. Alternatively, or additionally, the level of specific proteins encoded by a marker gene may be determined.

The levels of transcripts of specific marker genes can be accomplished by determining the amount of mRNA, or polynucleotides derived therefrom, present in a sample. Any method for determining RNA levels can be used. For example, RNA is isolated from a sample and separated on an agarose gel. The separated RNA is then transferred to a solid support, such as a filter. Nucleic acid probes representing one or more markers are then hybridized to the filter by northern hybridization, and the amount of marker-derived RNA is determined. Such determination can be visual, or machine-aided, for example, by use of a densitometer. Another method of determining RNA levels is by use of a dot-blot or a slot-blot. In this method, RNA, or nucleic acid derived therefrom, from a sample is labeled. The RNA or nucleic acid derived therefrom is then hybridized to a filter containing oligonucleotides derived from one or more marker genes, wherein the oligonucleotides are placed upon the filter at discrete, easily-identifiable locations. Hybridization, or lack thereof, of the labeled RNA to the filter-bound oligonucleotides is determined visually or by densitometer. Polynucleotides can be labeled using a radiolabel or a fluorescent (i.e., visible) label.

These examples are not intended to be limiting; other methods of determining RNA abundance are known in the art.

The levels of transcripts of particular marker genes may also be assessed by determining the level of the specific protein expressed from the marker genes. This can be accomplished, for example, by separation of proteins from a sample on a polyacrylamide gel, followed by identification of specific marker-derived proteins using antibodies in a western blot. Alternatively, proteins can be separated by two-dimensional gel electrophoresis systems. Two-dimensional gel electrophoresis is well-known in the art and typically involves isoelectric focusing along a first dimension followed by SDS-PAGE electrophoresis along a second dimension. See, e.g., Hames et al, 1990, GEL ELECTROPHORESIS OF PROTEINS: A PRACTICAL APPROACH, IRL Press, New York; Shevchenko et al., Proc. Nat'l Acad. Sci. USA 93:1440-1445 (1996); Sagliocco et al., Yeast 12:1519-1533 (1996); Lander, Science 274:536-539 (1996). The resulting electropherograms can be analyzed by numerous techniques, including mass spectrometric techniques, western blotting and immunoblot analysis using polyclonal and monoclonal antibodies.

Alternatively, marker-derived protein levels can be determined by constructing an antibody microarray in which binding sites comprise immobilized, preferably monoclonal, antibodies specific to a plurality of protein species encoded by the cell genome. Preferably, antibodies are present for a substantial fraction of the marker-derived proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array, and their binding is assayed with assays known in the art. Generally, the expression, and the level of expression, of proteins of diagnostic or prognostic interest can be detected through immunohistochemical staining of tissue slices or sections.

Finally, levels of transcripts of marker genes in a number of tissue specimens may be characterized using a “tissue array” (Kononen et al., Nat. Med 4(7):844-7 (1998)). In a tissue array, multiple tissue samples are assessed on the same microarray. The arrays allow in situ detection of RNA and protein levels; consecutive sections allow the analysis of multiple samples simultaneously.

Microarrays

In preferred embodiments, polynucleotide microarrays are used to measure expression so that the expression status of each of the markers above is assessed simultaneously. Generally, microarrays according to the invention comprise a plurality of markers informative for prognosis, or outcome determination, for a particular disease or condition, and, in particular, for individuals having specific combinations of genotypic or phenotypic characteristics of the disease or condition (i.e., that are prognosis-informative for a particular patient subset).

The invention also provides a microarray comprising for each of the plurality of genes listed in Table 1, one or more polynucleotide probes complementary and hybridizable to a sequence in said gene, wherein polynucleotide probes complementary and hybridizable to said genes constitute at least 50%, 60%, 70%, 80%, 90%, 95%, or 98% of the probes on said microarray. In a particular embodiment, the invention provides such a microarray wherein the plurality of genes comprises the 9 genes listed in Table 1. The microarray can be in a sealed container.

In specific embodiments, the invention provides polynucleotide arrays in which the prognosis markers identified for a particular patient subset comprise at least 50%, 60%, 70%, 80%, 85%, 90%, 95% or 98% of the probes on the array. In another specific embodiment, the microarray comprises a plurality of probes, wherein said plurality of probes comprise probes complementary and hybridizable to at least 75% of the prognosis-informative markers identified for a particular patient subset. Microarrays of the invention, of course, may comprise probes complementary and hybridizable to prognosis-informative markers for a plurality of the patient subsets, or for each patient subset, identified for a particular condition. In another embodiment, therefore, the microarray of the invention comprises a plurality of probes complementary and hybridizable to at least 75% of the prognosis-informative markers identified for each patient subset identified for the condition of interest, and wherein the probes, in total, are at least 50% of the probes on said microarray.

In yet another specific embodiment, the microarray is a commercially-available cDNA microarray that comprises probes to at least five markers identified by the methods described herein. Preferably, a commercially-available cDNA microarray comprises probes to all of the markers identified by the methods described herein as being informative for a patient subset for a particular condition.

The invention provides microarrays containing probes useful for the prognosis of colon cancer patients. In particular, the invention provides polynucleotide arrays comprising probes to a subset, or up to the full set of markers, in Table 1, which distinguish between patients with good and poor prognosis. In certain embodiments, therefore, the invention provides microarrays comprising probes for a plurality of the genes for which markers are listed in Table 1. In a specific embodiment, the microarray of the invention comprises all of the markers in Table 1. In other embodiments, the microarray of the invention contains each of the markers in Table 1. In another embodiment, the microarray contains all of the markers shown in Table 1.

In specific embodiments, the invention provides polynucleotide arrays in which the colon cancer prognosis markers described herein in Table 1 comprise at least 50%, 60%, 70%, 80%, 85%, 90%, 95% or 98% of the probes on said array. In another specific embodiment, the microarray comprises a plurality of probes, wherein said plurality of probes comprise probes complementary and hybridizable to transcripts of at least 75% of the genes for which markers are listed in Table 1.

In yet another specific embodiment, the microarray is a commercially-available cDNA microarray that comprises probes to at least five of the markers listed in Table 1. Preferably, a commercially-available cDNA microarray comprises all of the markers listed in Table 1. However, such a microarray may comprise probes to at least 2, 4 or 6 of the markers in Table 1, up to the maximum number of markers in Table 1, and may comprise probes to all of the markers in Table 1. In a specific embodiment of the microarrays used in the methods disclosed herein comprise probes to the markers that are all or a portion of Table 1 make up at least 50%, 60%, 70%, 80%, 90%, 95% or 98% of the probes on the microarray.

General methods pertaining to the construction of microarrays comprising the marker sets and/or subsets above are described in the following sections.

In a specific embodiment, the Affymetrix® Human Genome U133Plus2 (HG-U133) Set, consisting of two GeneChip® arrays, is used in accordance with known methods. The Human Genome U133 (HG-U133) Set contains almost 45,000 probe sets representing more than 39,000 transcripts derived from approximately 33,000 well-substantiated human genes. This set design uses sequences selected from GenBank®, dbEST, and RefSeq. The sequence clusters were created from the UniGene database (Build 133, Apr. 20, 2001). They were then refined by analysis and comparison with a number of other publicly available databases including the Washington University EST trace repository and the University of California, Santa Cruz Golden Path human genome database (April 2001 release).

In another embodiment, the HG-U133A array is used in accordance with the methods of the invention. The HG-U133A array includes representation of the RefSeq database sequences and probe sets related to sequences previously represented on the Human Genome U95Av2 array. The HG-U133B array contains primarily probe sets representing EST clusters. In another embodiment, the U133 Plus 2.0 GeneChip® is used in the invention. The U133 Plus 2.0 GeneChip® represents over 47,000 transcripts.

In another embodiment, a cDNA based microarray is used. In one embodiment, TIGR's 32,488-element spotted cDNA arrays is used. The TIGR cDNA array contains 31,872 human cDNAs representing 30,849 distinct transcripts: 23,936 unique TIGR TCs and 6,913 ESTs, 10 exogenous controls printed 36 times, and 4 negative controls printed 36-72 times.

Construction of Microarray

Microarrays are prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

The probe or probes used in the methods of the invention are preferably immobilized to a solid support which may be either porous or non-porous. For example, the probes of the invention may be polynucleotide sequences which are attached to a nitrocellulose or nylon membrane or filter covalently at either the 3′ or the 5′ end of the polynucleotide. Such hybridization probes are well known in the art (see, e.g., Sambrook et al., MOLECULAR CLONING—A LABORATORY MANUAL (2ND ED.), Vols. 1-3, Cold Spring Harbor Laboratory, Cold Spring Harbor, N.Y. (1989). Alternatively, the solid support or surface may be a glass or plastic surface. In a particularly preferred embodiment, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel.

In preferred embodiments, a microarray comprises a support or surface with an ordered array of binding (e.g., hybridization) sites or “probes” each representing one of the markers described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). In preferred embodiments, each probe is covalently attached to the solid support at a single site.

Microarrays can be made in a number of ways, of which several are described below. However produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. The microarrays are preferably small, e.g., between 1 cm² and 25 cm², between 12 cm² and 13 cm², or 3 cm². However, larger arrays are also contemplated and may be preferable, e.g., for use in screening arrays. Preferably, a given binding site or unique set of binding sites in the microarray will specifically bind (e.g., hybridize) to the product of a single gene in a cell (e.g., to a specific mRNA, or to a specific cDNA derived therefrom). However, in general, other related or similar sequences will cross hybridize to a given binding site.

The microarrays of the present invention include one or more test probes, each of which has a polynucleotide sequence that is complementary to a subsequence of RNA or DNA to be detected. Preferably, the position of each probe on the solid surface is known. Indeed, the microarrays are preferably positionally addressable arrays. Specifically, each probe of the array is preferably located at a known, predetermined position on the solid support such that the identity (i.e., the sequence) of each probe can be determined from its position on the array (i.e., on the support or surface).

According to the invention, the microarray is an array (i.e., a matrix) in which each position represents one of the markers described herein. For example, each position can contain a DNA or DNA analogue based on genomic DNA to which a particular RNA or cDNA transcribed from that genetic marker can specifically hybridize. The DNA or DNA analogue can be, e.g., a synthetic oligomer or a gene fragment. In one embodiment, probes representing each of the markers is present on the array. In a preferred embodiment, the array comprises probes for each of the markers listed in Table 1.

Preparing Probes for Microarrays

As noted above, the “probe” to which a particular polynucleotide molecule specifically hybridizes according to the invention contains a complementary genomic polynucleotide sequence. The probes of the microarray preferably consist of nucleotide sequences of no more than 1,000 nucleotides. In some embodiments, the probes of the array consist of nucleotide sequences of 10 to 1,000 nucleotides. In a preferred embodiment, the nucleotide sequences of the probes are in the range of 10-200 nucleotides in length and are genomic sequences of a species of organism, such that a plurality of different probes is present, with sequences complementary and thus capable of hybridizing to the genome of such a species of organism, sequentially tiled across all or a portion of such genome. In other specific embodiments, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, and most preferably are 60 nucleotides in length.

The probes may comprise DNA or DNA “mimics” (e.g., derivatives and analogues) corresponding to a portion of an organism's genome. In another embodiment, the probes of the microarray are complementary RNA or RNA mimics DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates.

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically each probe on the microarray will be between 10 bases and 50,000 bases, usually between 300 bases and 1,000 bases in length. PCR methods are well known in the art, and are described, for example, in Innis et al., eds., PCR PROTOCOLS: A GUIDE TO METHODS AND APPLICATIONS, Academic Press Inc., San Diego, Calif. (1990). It will be apparent to one skilled in the art that controlled robotic systems are useful for isolating and amplifying nucleic acids.

An alternative, preferred means for generating the polynucleotide probes of the microarray is by synthesis of synthetic polynucleotides or oligonucleotides, e.g., using N-phosphonate or phosphoramidite chemistries (Froehler et al., Nucleic Acid Res. 14:5399-5407 (1986); McBride et al., Tetrahedron Lett. 24:246-248 (1983)). Synthetic sequences are typically between about 10 and about 500 bases in length, more typically between about 20 and about 100 bases, and most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., Nature 363:566-568 (1993); U.S. Pat. No. 5,539,083).

Probes are preferably selected using an algorithm that takes into account binding energies, base composition, sequence complexity, cross-hybridization binding energies, and secondary structure. See Friend et al., International Patent Publication WO 01/05935, published Jan. 25, 2001; Hughes et al., Nat. Biotech. 19:342-7 (2001).

A skilled artisan will also appreciate that positive control probes, e.g., probes known to be complementary and hybridizable to sequences in the target polynucleotide molecules, and negative control probes, e.g., probes known to not be complementary and hybridizable to sequences in the target polynucleotide molecules, should be included on the array. In one embodiment, positive controls are synthesized along the perimeter of the array. In another embodiment, positive controls are synthesized in diagonal stripes across the array. In still another embodiment, the reverse complement for each probe is synthesized next to the position of the probe to serve as a negative control. In yet another embodiment, sequences from other species of organism are used as negative controls or as “spike-in” controls.

Attaching Probes to the Solid Surface

The probes are attached to a solid support or surface, which may be made, e.g., from glass, plastic (e.g., polypropylene, nylon), polyacrylamide, nitrocellulose, gel, or other porous or nonporous material. A preferred method for attaching the nucleic acids to a surface is by printing on glass plates, as is described generally by Schena et al, Science 270:467-470 (1995). This method is especially useful for preparing microarrays of cDNA (See also, DeRisi et al, Nature Genetics 14:457-460 (1996); Shalon et al., Genome Res. 6:639-645 (1996); and Schena et al., Proc. Natl. Acad. Sci. U.S.A. 93:10539-11286 (1995)).

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). When these methods are used, oligonucleotides (e.g., 60-mers) of known sequence are synthesized directly on a surface such as a derivatized glass slide. Usually, the array produced is redundant, with several oligonucleotide molecules per RNA.

Other methods for making microarrays, e.g., by masking (Maskos and Southern, 1992, Nuc. Acids. Res. 20:1679-1684), may also be used. In principle, and as noted supra, any type of array, for example, dot blots on a nylon hybridization membrane (see Sambrook et al., MOLECULAR CLONING—A LABORATORY MANUAL (2ND ED.), Vols. 1-3, Cold Spring Harbor Laboratory, Cold Spring Harbor, N.Y. (1989)) could be used. However, as will be recognized by those skilled in the art, very small arrays will frequently be preferred because hybridization volumes will be smaller.

In one embodiment, the arrays of the present invention are prepared by synthesizing polynucleotide probes on a support. In such an embodiment, polynucleotide probes are attached to the support covalently at either the 3′ or the 5′ end of the polynucleotide.

In a particularly preferred embodiment, microarrays of the invention are manufactured by means of an ink jet printing device for oligonucleotide synthesis, e.g., using the methods and systems described by Blanchard in U.S. Pat. No. 6,028,189; Blanchard et al., 1996, Biosensors and Bioelectronics 11:687-690; Blanchard, 1998, in Synthetic DNA Arrays in Genetic Engineering, Vol. 20, J. K. Setlow, Ed., Plenum Press, New York at pages 111-123. Specifically, the oligonucleotide probes in such microarrays are preferably synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes). Microarrays manufactured by this ink-jet method are typically of high density, preferably having a density of at least about 2,500 different probes per 1 cm². The polynucleotide probes are attached to the support covalently at either the 3′ or the 5′ end of the polynucleotide.

Target Labeling and Hybridization to Microarrays

The polynucleotide molecules which may be analyzed by the present invention (the “target polynucleotide molecules”) may be from any clinically relevant source, but are expressed RNA or a nucleic acid derived therefrom (e.g., cDNA or amplified RNA derived from cDNA that incorporates an RNA polymerase promoter), including naturally occurring nucleic acid molecules, as well as synthetic nucleic acid molecules. In one embodiment, the target polynucleotide molecules comprise RNA, including, but by no means limited to, total cellular RNA, poly(A)⁺ messenger RNA (mRNA) or fraction thereof, cytoplasmic mRNA, or RNA transcribed from cDNA (i.e., cRNA; see, e.g., Linsley & Schelter, U.S. patent application Ser. No. 09/411,074, filed Oct. 4, 1999, or U.S. Pat. Nos. 5,545,522, 5,891,636, or 5,716,785). Methods for preparing total and poly(A)⁺ RNA are well known in the art, and are described generally, e.g., in Sambrook et al., MOLECULAR CLONING—A LABORATORY MANUAL (2ND ED.), Vols. 1-3, Cold Spring Harbor Laboratory, Cold Spring Harbor, N.Y. (1989). In one embodiment, RNA is extracted from cells of the various types of interest in this invention using guanidinium thiocyanate lysis followed by CsCl centrifugation (Chirgwin et al., 1979, Biochemistry 18:5294-5299). In another embodiment, total RNA is extracted using a silica gel-based column, commercially available examples of which include RNeasy (Qiagen, Valencia, Calif.) and StrataPrep (Stratagene, La Jolla, Calif.). In an alternative embodiment, which is preferred for S. cerevisiae, RNA is extracted from cells using phenol and chloroform, as described in Ausubel et al., eds., 1989, CURRENT PROTOCOLS IN MOLECULAR BIOLOGY, Vol. III, Green Publishing Associates, Inc., John Wiley & Sons, Inc., New York, at pp. 13.12.1-13.12.5). Poly(A)⁺ RNA can be selected, e.g., by selection with oligo-dT cellulose or, alternatively, by oligo-dT primed reverse transcription of total cellular RNA. In one embodiment, RNA can be fragmented by methods known in the art, e.g., by incubation with ZnCl₂, to generate fragments of RNA. In another embodiment, the polynucleotide molecules analyzed by the invention comprise cDNA, or PCR products of amplified RNA or cDNA.

In one embodiment, total RNA, mRNA, or nucleic acids derived therefrom, is isolated from a sample taken from a colorectal cancer patient. Target polynucleotide molecules that are poorly expressed in particular cells may be enriched using normalization techniques (Bonaldo et al., 1996, Genome Res. 6:791-806).

As described above, the target polynucleotides are detectably labeled at one or more nucleotides. Any method known in the art may be used to detectably label the target polynucleotides. Preferably, this labeling incorporates the label uniformly along the length of the RNA, and more preferably, the labeling is carried out at a high degree of efficiency. One embodiment for this labeling uses oligo-dT primed reverse transcription to incorporate the label; however, conventional methods of this method are biased toward generating 3′ end fragments. Thus, in a preferred embodiment, random primers (e.g., 9-mers) are used in reverse transcription to uniformly incorporate labeled nucleotides over the full length of the target polynucleotides. Alternatively, random primers may be used in conjunction with PCR methods or T7 promoter-based in vitro transcription methods in order to amplify the target polynucleotides.

In a preferred embodiment, the detectable label is a luminescent label. For example, fluorescent labels, bioluminescent labels, chemiluminescent labels, and colorimetric labels may be used in the present invention. In a highly preferred embodiment, the label is a fluorescent label, such as a fluorescein, a phosphor, a rhodamine, or a polymethine dye derivative. Examples of commercially available fluorescent labels include, for example, fluorescent phosphoramidites such as FluorePrime (Amersham Pharmacia, Piscataway, N.J.), Fluoredite (Millipore, Bedford, Mass.), FAM (ABI, Foster City, Calif.), and Cy3 or Cy5 (Amersham Pharmacia, Piscataway, N.J.). In another embodiment, the detectable label is a radiolabeled nucleotide.

In a further preferred embodiment, target polynucleotide molecules from a patient sample are labeled differentially from target polynucleotide molecules of a reference sample. The reference can comprise target polynucleotide molecules from normal tissue samples (i.e., tissues from those not afflicted with colorectal cancer).

Nucleic acid hybridization and wash conditions are chosen so that the target polynucleotide molecules specifically bind or specifically hybridize to the complementary polynucleotide sequences of the array, preferably to a specific array site, wherein its complementary DNA is located.

Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self complementary sequences.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook et al., MOLECULAR CLONING—A LABORATORY MANUAL (2ND ED.), Vols. 1-3, Cold Spring Harbor Laboratory, Cold Spring Harbor, N.Y. (1989), and in Ausubel et al., CURRENT PROTOCOLS IN MOLECULAR BIOLOGY, vol. 2, Current Protocols Publishing, New York (1994). Typical hybridization conditions for the cDNA microarrays of Schena et al. are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Schena et al., Proc. Natl. Acad. Sci. U.S.A. 93:10614 (1993)). Useful hybridization conditions are also provided in, e.g., Tijessen, 1993, HYBRIDIZATION WITH NUCLEIC ACID PROBES, Elsevier Science Publishers B.V.; and Kricka, 1992, NONISOTOPIC DNA PROBE TECHNIQUES, Academic Press, San Diego, Calif.

Particularly preferred hybridization conditions include hybridization at a temperature at or near the mean melting temperature of the probes (e.g., within 51° C., more preferably within 21° C.) in 1 M NaCl, 50 mM MES buffer (pH 6.5), 0.5% sodium sarcosine and 30% formamide.

Signal Detection and Data Analysis

When fluorescently labeled gene products are used, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously (see Shalon et al., 1996, “A DNA microarray system for analyzing complex DNA samples using two-color fluorescent probe hybridization,” Genome Research 6:639-645, which is incorporated by reference in its entirety for all purposes). In a preferred embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., Genome Res. 6:639-645 (1996), and in other references cited herein. Alternatively, the fiber-optic bundle described by Ferguson et al., Nature Biotech. 14:1681-1684 (1996), may be used to monitor mRNA abundance levels at a large number of sites simultaneously.

Other Assays for Detecting and Quantifying RNA

In addition to microarrays such as those described above any technique known to one of skill for detecting and measuring RNA can be used in accordance with the methods of the invention. Non-limiting examples of techniques include Northern blotting, nuclease protection assays, RNA fingerprinting, polymerase chain reaction, ligase chain reaction, Qbeta replicase, isothermal amplification method, strand displacement amplification, transcription based amplification systems, nuclease protection (SI nuclease or RNAse protection assays), SAGE as well as methods disclosed in International Publication Nos. WO 88/10315 and WO 89/06700, and International Applications Nos. PCT/US87/00880 and PCT/US89/01025.

A standard Northern blot assay can be used to ascertain an RNA transcript size, identify alternatively spliced RNA transcripts, and the relative amounts of mRNA in a sample, in accordance with conventional Northern hybridization techniques known to those persons of ordinary skill in the art. In Northern blots, RNA samples are first separated by size via electrophoresis in an agarose gel under denaturing conditions. The RNA is then transferred to a membrane, crosslinked and hybridized with a labeled probe. Nonisotopic or high specific activity radiolabeled probes can be used including random-primed, nick-translated, or PCR-generated DNA probes, in vitro transcribed RNA probes, and oligonucleotides. Additionally, sequences with only partial homology (e.g., cDNA from a different species or genomic DNA fragments that might contain an exon) may be used as probes. The labeled probe, e.g., a radiolabelled cDNA, either containing the full-length, single stranded DNA or a fragment of that DNA sequence may be at least 20, at least 30, at least 50, or at least 100 consecutive nucleotides in length. The probe can be labeled by any of the many different methods known to those skilled in this art. The labels most commonly employed for these studies are radioactive elements, enzymes, chemicals that fluoresce when exposed to ultraviolet light, and others. A number of fluorescent materials are known and can be utilized as labels. These include, but are not limited to, fluorescein, rhodamine, auramine, Texas Red, AMCA blue and Lucifer Yellow. A particular detecting material is anti-rabbit antibody prepared in goats and conjugated with fluorescein through an isothiocyanate. Proteins can also be labeled with a radioactive element or with an enzyme. The radioactive label can be detected by any of the currently available counting procedures. Non-limiting examples of isotopes include ³H, ¹⁴C, ³²F, ³⁵S, ³⁶Ci, ⁵¹Cr, C ⁵⁷Co, ⁵⁸Co, ⁵⁹Fe, ⁹⁰Y, ¹²⁵I, ¹³¹I, and ¹⁸⁶Re. Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Any enzymes known to one of skill in the art can be utilized. Examples of such enzymes include, but are not limited to, peroxidase, beta-D-galactosidase, urease, glucose oxidase plus peroxidase and alkaline phosphatase. U.S. Pat. Nos. 3,654,090, 3,850,752, and 4,016,043 are referred to by way of example for their disclosure of alternate labeling material and methods.

Nuclease protection assays (including both ribonuclease protection assays and S1 nuclease assays) can be used to detect and quantitate specific mRNAs. In nuclease protection assays, an antisense probe (labeled with, e.g., radiolabeled or nonisotopic) hybridizes in solution to an RNA sample. Following hybridization, single-stranded, unhybridized probe and RNA are degraded by nucleases. An acrylamide gel is used to separate the remaining protected fragments. Typically, solution hybridization is more efficient than membrane-based hybridization, and it can accommodate up to 100 μg of sample RNA, compared with the 20-30 μg maximum of blot hybridizations.

The ribonuclease protection assay, which is the most common type of nuclease protection assay, requires the use of RNA probes. Oligonucleotides and other single-stranded DNA probes can only be used in assays containing S1 nuclease. The single-stranded, antisense probe must typically be completely homologous to target RNA to prevent cleavage of the probe:target hybrid by nuclease.

Serial Analysis Gene Expression (SAGE), which is described in e.g., Velculescu et al., 1995, Science 270:484-7; Carulli, et al., 1998, Journal of Cellular Biochemistry Supplements 30/31:286-96, can also be used to determine RNA abundances in a cell sample.

Quantitative reverse transcriptase PCR (qRT-PCR) can also be used to determine the expression profiles of marker genes (see, e.g., U.S. Patent Application Publication No. 2005/0048542A1). The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™. Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system includes software for running the instrument and for analyzing the data.

5.#8242;-Nuclease assay data are initially expressed as Ct, or the threshold cycle. Fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

Detection and Quantification of Protein

Measurement of the translational state may be performed according to several methods. For example, whole genome monitoring of protein (e.g., the “proteome,”) can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to the action of a drug of interest. Methods for making monoclonal antibodies are well known (see, e.g., Harlow and Lane, 1988, Antibodies: A Laboratory Manual, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array and their binding is assayed with assays known in the art.

Immunoassays known to one of skill in the art can be used to detect and quantify protein levels. For example, ELISAs can be used to detect and quantify protein levels. ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. In a preferred embodiment, an ELISA may be performed by coating a high binding 96-well microtiter plate (Costar) with 2 μg/ml of rhu-IL-9 in PBS overnight. Following three washes with PBS, the plate is incubated with three-fold serial dilutions of Fab at 25° C. for 1 hour. Following another three washes of PBS, 1 μg/ml anti-human kappa-alkaline phosphatase-conjugate is added and the plate is incubated for 1 hour at 25° C. Following three washes with PBST, the alkaline phosphatase activity is determined in 50 μl/AMP/PPMP substrate. The reactions are stopped and the absorbance at 560 nm is determined with a VMAX microplate reader. For further discussion regarding ELISAs see, e.g., Ausubel et al, eds, 1994, Current Protocols in Molecular Biology, Vol. 1, John Wiley & Sons, Inc., New York at 11.2.1.

Protein levels may be determined by Western blot analysis. Further, protein levels as well as the phosphorylation of proteins can be determined by immunoprecitation followed by Western blot analysis Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1 to 4 hours) at 40° C., adding protein A and/or protein G sepharose beads to the cell lysate, incubating for about an hour or more at 40° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, incubating the membrane in blocking solution (e.g., PBS with 3% BSA or non-fat milk), washing the membrane in washing buffer (e.g., PBS-Tween 20), incubating the membrane with primary antibody (the antibody of interest) diluted in blocking buffer, washing the membrane in washing buffer, incubating the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

Protein expression levels can also be separated by two-dimensional gel electrophoresis systems. Two-dimensional gel electrophoresis is well-known in the art and typically involves iso-electric focusing along a first dimension followed by SDS-PAGE electrophoresis along a second dimension. See, e.g., Hames et al., 1990, Gel Electrophoresis of Proteins: A Practical Approach, IRL Press, New York; Shevchenko et al., 1996, Proc. Natl. Acad. Sci. USA 93:1440-1445; Sagliocco et al., 1996, Yeast 12:1519-1533; Lander, 1996, Science 274:536-539. The resulting electropherograms can be analyzed by numerous techniques, including mass spectrometric techniques, Western blotting and immunoblot analysis using polyclonal and monoclonal antibodies, and internal and N-terminal micro-sequencing.

Determining Therapeutic Regimens for Patients

The benefit of adjuvant chemotherapy for colorectal cancer appears limited to patients with Dukes stage C disease where the cancer has metastasized to lymph nodes at the time of diagnosis. For this reason, the clinicopathological Dukes' staging system is critical for determining how adjuvant therapy is administered. Unfortunately, as noted above, Dukes' staging is not very accurate in predicting overall survival and thus its application likely results in the treatment of a large number of patients to benefit an unknown few. Alternatively, there are a number of patients who would benefit from therapy that do not receive it based on the Dukes' staging system.

Thus, the methods of the prognosis prediction can be used for determining whether a colorectal cancer patient may benefit from chemotherapy. In one embodiment, the invention provides a method for determining whether a colorectal cancer patient should be treated with chemotherapy, comprising (a) classifying the patient as having a good prognosis or a poor prognosis using a method as described herein; and (b) determining that said patient's predicted survival time favors treatment of the patient with chemotherapy if said patient is classified as having a poor prognosis. In another embodiment, the methods are used in conjunction with Dukes staging. For example, the prognosis methods of the invention can be used to identify those Dukes' stage B and C cases for which chemotherapy may be beneficial.

If a patient is determined to be one likely to benefit from chemotherapy, a suitable chemotherapy may be prescribed for the patient. Chemotherapy can be performed using any one or a combination of the anti-cancer drugs known in the art, including but not limited to any topoisomerase inhibitor, DNA binding agent, anti-metabolite, ionizing radiation, or a combination of two or more of such known DNA damaging agents.

### EXAMPLE

The following example is presented by way of illustration of the present invention, and is not intended to limit the present invention in any way.

The inventors used gene expression data derived from a prospective clinical randomized two arm Phase II chemotherapy trial for first line metastatic colorectal cancer to produce a gene signature that separates patients likely to respond (responders) to standard therapies from those that may not respond (non-responders). The trial involved more than 85 patients treated with one of two types of standard chemotherapy for colorectal cancer: (a) XELOX/AVASTIN and (b) XELIRI/AVASTIN.

The inventors combined the data from both arms of the trial to look for responders and non-responders to both standard types of therapy. More than 90% of patients with metastatic colorectal cancer will receive one of these regimens in standard practice today. A liver core biopsy was obtained from each patient's liver metastasis prior to initiation of therapy. Biopsies were used to extract derivative RNA that was subsequently used to perform whole genome microarray analysis on Affymetrix U133PLUS2.0 GeneChips. Derivative gene expression profiles were used to identify key genes and gene families linked to response vs. non-response of colorectal cancer to the standard colorectal cancer regimens. An initial set of genes significantly over- or under-expressed between responder and non-responder groups were identified by using a t-test (P<0.01). This set was refined by excluding those genes which also exhibited a significant frequency of Type I errors using an F-test. Using this approach, the inventors have identified 9 key genes using the clinical trial raw data that cleanly distinguish responder from non-responder patients. These genes include DNA repair, apoptosis, and angiogenesis pathways.

Genes have also been identified, in the attached tables, that include a broader range of genes primarily involved in DNA repair pathways and apoptosis pathways that the 9 genes reside in. One of the genes in the signature also relates to the Notch pathway and may be a strong predictor for response to Notch inhibitors.

To employ the data obtained from expression profile experiments for prediction of response to therapy, the inventors utilized experiments conducted on Affymetrix U133Plus2 GeneChips, and processed the data using the MASS algorithm. Average expression values for 3 genes involved in apoptosis downstream of DNA damage (DD) were calculated using the probes identified in Table 1. Composite values for this group ranged from 844 to 1512 in the samples obtained from non-responders, while they ranged from 2035 to 3122 in samples obtained from responders (Table 2). A similar composite score was calculated for VEGF, ITGB6, and KRT80 and scaled to allow comparison with the damage/apoptosis genes identified above (Table 3).

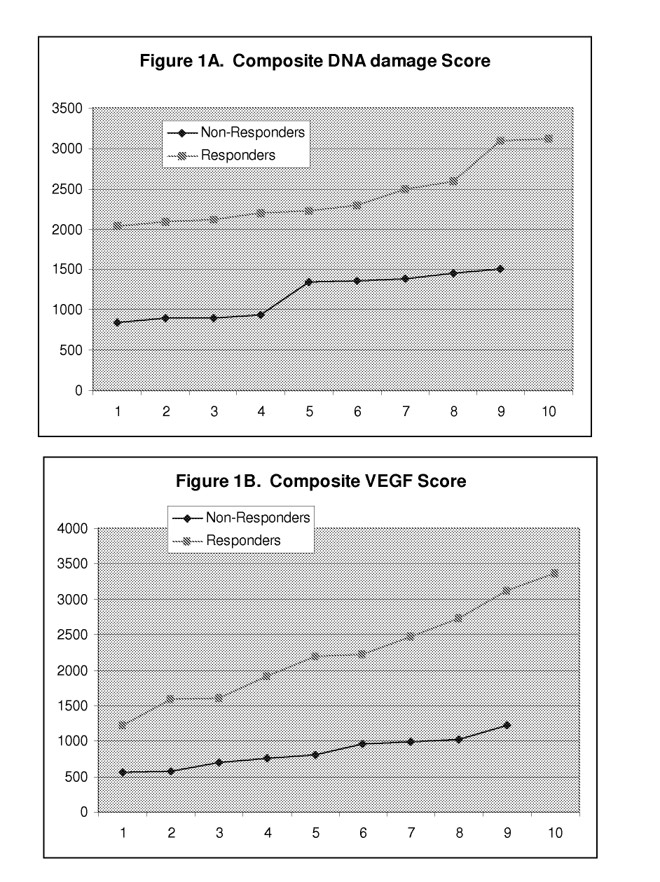

The distribution of both composite values are plotted in FIGS. 1A and 1B. Based on this data, responders can be identified as having composite DD scores over 1500 and composite VEGF scores over 1000. Since MASS allows for independent normalization of GeneChip data, it is proposed that responder signatures may be determined by conducting expression profiling on this platform and utilizing the MASS generated output.

TABLE 1
Apoptosis/DNA damage Genes

| Affymetrix Probe ID | Gene ID |
|---|---|
| 224825_at | DNTTIP1 |
| 234942_s_at | DNTTIP1 |
| 201170_s_at | BHLHB2 |
| 201169_s_at | BHLHB2 |
| 208861_s_at | ATRX |
| 208859_s_at | ATRX |

TABLE 2
Composite DNA Damage Score

| Non-Responders | Responders |
|---|---|
| 844 | 2035 |
| 889 | 2094 |
| 891 | 2123 |
| 934 | 2194 |
| 1345 | 2229 |
| 1359 | 2289 |
| 1387 | 2492 |
| 1453 | 2595 |
| 1512 | 3093 |
| | 3122 |

TABLE 3
Composite VEGF Score

| Non-Responders | Responders |
|---|---|
| 556 | 1221 |
| 572 | 1584 |
| 698 | 1612 |
| 751 | 1912 |
| 807 | 2192 |
| 958 | 2223 |
| 985 | 2474 |
| 1013 | 2734 |
| 1223 | 3116 |
| | 3372 |

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A method of determining a therapeutic regimen for a colorectal cancer patient, comprising classifying said patient as having a good prognosis or a poor prognosis using measurements of a plurality of gene products in a cell sample taken from said patient, said gene products being respectively products of at least 1 of the genes listed in Table 1 or respective functional equivalents thereof, wherein said good prognosis predicts the patient's response to chemotherapy, and said poor prognosis predicts the patient's non-responsiveness to chemotherapy.

2. The method of claim 1, wherein said plurality of gene products are of at least 5 of the genes listed in Table 1.

3. The method of claim 1, further comprising obtaining said marker profile by a method comprising measuring said plurality of gene products in said tumor cell sample.

4. The method of claim 1, wherein said classifying is carried out by a method comprising using a prognosis predictor, wherein said prognosis predictor receives an input comprising said measurements and provides an output comprising data indicating a good prognosis or a poor prognosis.

5. The method of claim 4, wherein said prognosis predictor is trained with training data from a plurality of colorectal cancer patients, wherein said training data comprise for each of said plurality of colorectal cancer patients (a) measurements of said plurality of gene products in a cell sample taken from said patient and (b) information with respect to whether survival for said time period occurred or not.

6. The method of claim 1, wherein each of said gene products is a gene transcript.

7. The method of claim 6, wherein measurement of each said gene transcript is obtained by a method comprising contacting a positionally-addressable microarray with nucleic acids from said cell sample or nucleic acids derived therefrom under hybridization conditions, and detecting the amount of hybridization that occurs, said microarray comprising one or more polynucleotide probes complementary to a hybridizable sequence of each said gene transcript.

8. The method of claim 7, wherein said microarray is selected from the group consisting of cDNA microarray, ink-jet synthesized microarray, and oligonucleotide microarray.

9. The method of claim 1, wherein each of said plurality of gene products is a protein.

10. The method of claim 1, wherein said patient has an increased level of a gene product relative to the average level of said gene product in patients without colorectal cancer.
